(19) United States
(12) Patent Application Publication
(10) Pub. No.: US 2008/0058535 A1
(43) Pub. Date: Mar. 6, 2008

(54) METHODS AND COMPOSITIONS FOR PRODUCTION, FORMULATION AND USE OF 1 ARYL-3-AZABICYCLO[3.1.0]HEXANES

(76) Inventors: Zhengming Chen, Belle Mead, NJ (US); Phil SKOLNICK, Edgewater, NJ (US)

Correspondence Address: BLACK LOWE & GRAHAM PLLC;Suite 4800, 701 Fifth Avenue, Seattle, WA, 98104, US

(21) Appl. No.: 11/782,400

(22) Filed: Jul. 24, 2007

Related U.S. Application Data
(60) Provisional application No. 60/833,438, filed on Jul. 25, 2006.

(51) Int. Cl. C07D 209/52; C07C 211/17; C07C 255/46

(52) U.S. Cl. 548/512; 548/515; 558/426; 564/442

(57) ABSTRACT

The invention provides novel compositions and methods of making (−)-1-(3,4-dichlorophenyl)-3-azabicyclo[3.1.0]hexane and other 1-aryl-3-azabicyclo[3.1.0]hexanes, including synthetic methods that form novel intermediate compounds of the invention for producing)-(−)-1-(3,4-dichlorophenyl)-3-azabicyclo[3.1.0]hexane and other 1-aryl-3-azabicyclo[3.1.0]hexanes and pharmaceutically acceptable salts thereof.

### REFERENCE TO RELATED APPLICATIONS

This application is related to and claims the benefit of U.S. Provisional Application 60/833,438 filed Jul. 25, 2006. This application also claims priority to application Ser. No. 11/371,178 filed Mar. 7, 2006, which is related to and claims the benefit of Provisional Applications 60/661,662, filed on Mar. 8, 2005 and 60/701,562 filed on Jul. 22, 2005, and application Ser. No. 11/493,431 filed on Jul. 25, 2006, which is related to and claims the benefit of U.S. Provisional Application 60/703,364 filed on Jul. 27, 2005. The disclosures of all of the foregoing applications are incorporated by reference herein in their entirety.

### TECHNICAL FIELD

The present invention relates to methods and intermediates for preparing 1-aryl-3-azabicyclo[3.1.0]hexanes, including chiral 1-aryl-3-azabicyclo[3.1.0]hexanes.

### BACKGROUND OF THE INVENTION

The compound (−)-1-(3,4-dichlorophenyl)-3-azabicyclo[3.1.0]hexane and its pharmaceutically acceptable salts have been previously described as agents for treating or preventing a disorder alleviated by inhibiting dopamine reuptake, such as depression (See, U.S. Pat. Nos. 6,569,887 and 6,716,868). However, available methods for synthesizing (−)-1-(3,4-dichlorophenyl)-3-azabicyclo[3.1.0]hexanes and other 1-aryl-3-azabicyclo[3.1.0]hexanes are presently limited.

U.S. Pat. No. 4,231,935 (Example 37) describes the synthesis of racemic (±)-1-(3,4-dichlorophenyl)-3-azabicyclo[3.1.0]hexane hydrochloride according to the following scheme.

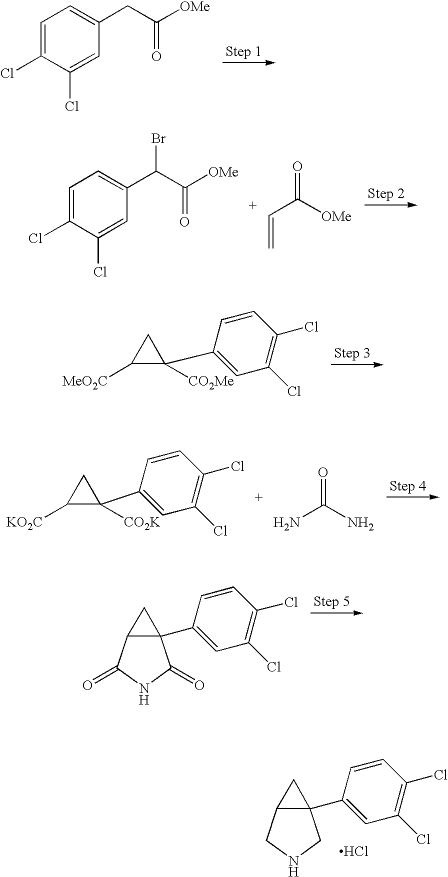

U.S. Pat. Nos. 6,569,887 and 6,716,868 describe the preparation of (−)-1-(3,4-dichlorophenyl)-3-azabicyclo[3.1.0]hexane by resolution of racemic (±)-1-(3,4-dichlorophenyl)-3-azabicyclo[3.1.0]hexane hydrochloride using a chiral polysaccharide stationary phase.

The foregoing methods provide limited tools for producing (−)-1-(3,4-dichlorophenyl)-3-azabicyclo[3.1.0]hexane and other 1-aryl-3-azabicyclo[3.1.0]hexanes, underscoring a need for additional methods and compositions to produce the compounds.

It is therefore an object of the present invention to provide novel compounds and synthetic methods for the preparation of (−)-1-(3,4-dichlorophenyl)-3-azabicyclo[3.1.0]hexane and other 1-aryl-3-azabicyclo[3.1.0]hexanes and their pharmaceutically acceptable salts.

### SUMMARY OF THE INVENTION

The present invention fulfills these needs and satisfies additional objects and advantages by providing novel synthetic processes for the production of (−)-1-(3,4-dichlorophenyl)-3-azabicyclo[3.1.0]hexane and other chiral 1-aryl-3-azabicyclo[3.1.0]hexanes.

In certain embodiments, the present invention provides methods for making (1S,5R)-(−)-1-(3,4-dichlorophenyl)-3-azabicyclo[3.1.0]hexane comprising:
(a) reacting a compound of the following formula (i), 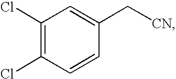
with (R)-(−)-epichlorohydrin to produce a compound of the following formula (ii), 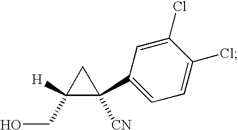
(b) reducing the compound of the formula (ii) to produce a compound of the following formula (iii), 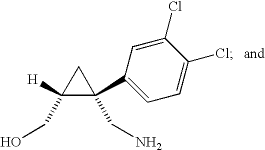
(c) causing cyclization of the compound of formula (iii) to produce (1S,5R)-(−)-1-(3,4-dichlorophenyl)-3-azabicyclo[3.1.0]hexane.

In additional embodiments, the present invention provides methods for making (1S,5R)-(−)-1-(3,4-dichlorophenyl)-3-azabicyclo[3.1.0]hexane comprising:
(a) reacting a compound of the following formula (i), 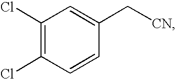
with (R)-(−)-epichlorohydrin to produce a compound of the following formula (ii), 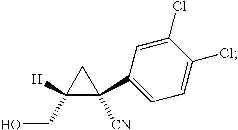
(b) reducing the compound of the formula (ii) to produce a compound of the following formula (iii), 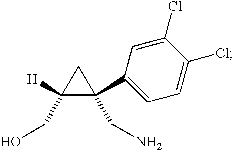
(c) reacting the compound of the formula (iii) with (Boc)₂O to produce a compound of the following formula (iv), 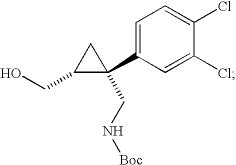
(d) deprotecting and causing cyclization of the compound of the formula (iv) to produce a compound of the following formula (v) 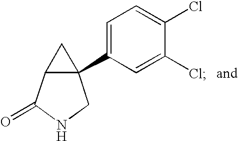
(e) reducing the compound of the formula (v) to produce (1S,5R)-(−)-1-(3,4-dichlorophenyl)-3-azabicyclo[3.1.0]hexane.

In further embodiments, the present invention provides methods of making 1-aryl-3-azabicyclo[3.1.0]hexanes of the following formula I, 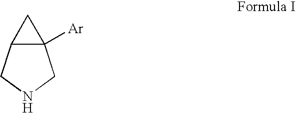
wherein Ar is a phenyl or napthyl group which is either unsubstituted or substituted with one or more substituents independently selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, comprising the steps of:
(a) reacting a compound of the following formula (vi), 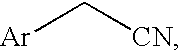
wherein Ar is defined as above, with epichlohydrin or an enantiomer thereof, to produce a compound of the following formula (vii), 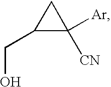
or an enantiomer or diastereomer thereof;
(b) oxidizing the compound of formula (vii) to produce a compound of the following formula (iii), 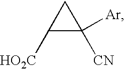
or an enantiomer or diastereomer thereof;
(c) causing esteration of the compound of formula (viii) to produce a compound of the following formula (ix), 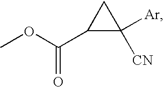
or an enantiomer or diastereomer thereof; and
(d) reducing the compound of formula (ix) to produce the 1-aryl-3-azabicyclo[3.1.0]hexane, or an enantiomer or diastereomer thereof.

In other embodiments, the present invention provides a method of making 1-(3,4-dichlorophenyl)-3-azabicyclo[3.1.0]hexane comprising:
(a) reacting a compound of the following formula (i), 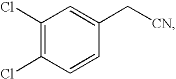
with epichlohydrin or an enantiomer thereof, to produce a compound of the following formula (x), 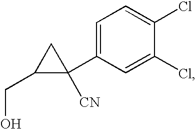
or an enantiomer or diastereomer thereof;

(b) oxidizing the compound of formula (x) to produce a compound of the following formula (xi), 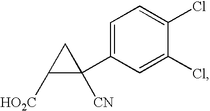
or an enantiomer or diastereomer thereof;
(c) causing esteration of the compound of formula (xi) to produce a compound of the following formula (xii), 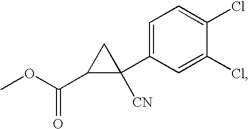
or an enantiomer or diastereomer thereof;
(d) reducing the compound of formula (xii) in the presence of a Raney nickel catalyst to produce a compound of the following formula (xiii), 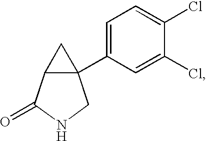
or an enantiomer or diastereomer thereof; and
(e) reducing the compound of formula (xiii) to produce the 1-(3,4-dichlorophenyl)-3-azabicyclo[3.1.0]hexane, or an enantiomer or diastereomer thereof.

The present invention also provides useful compounds for the production of (1S,5R)-(−)-1-(3,4-dichlorophenyl)-3-azabicyclo[3.1.0]hexane and other chiral 1-aryl-1-3-azabicyclo[3.1.0]hexanes.

In certain embodiments, the present invention provides a compound selected from the group consisting of compounds having the following formulas:

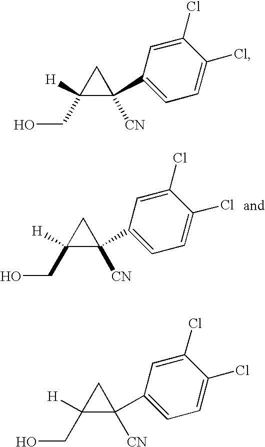

In additional embodiments, the present invention provides a compound selected from the group consisting of compounds having the following formulas and pharmaceutically acceptable salts thereof:

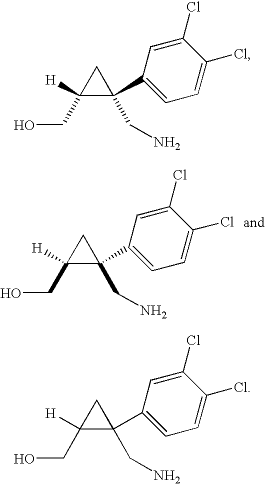

In further embodiments, the present invention provides a compound selected from the group consisting of compounds having the following formulas and pharmaceutically acceptable salts thereof: 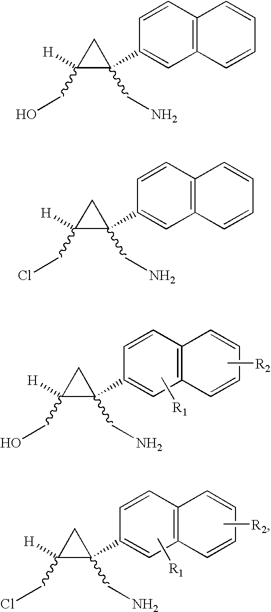
wherein R₁ and R₂ are independently selected from hydrogen, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

In other embodiments, the present invention provides a compound selected from the group consisting of compounds having the following formulas and pharmaceutically acceptable salts thereof: 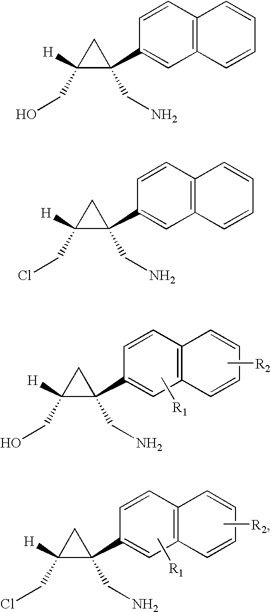
wherein R₁ and R₂ are independently selected from hydrogen, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

### DETAILED DESCRIPTION OF THE INVENTION

The present invention provides novel compounds and synthetic processes for the production of (−)-1-(3,4-dichlorophenyl)-3-azabicyclo[3.1.0]hexane and other 1-aryl-3-azabicyclo[3.1.0]hexanes.

Reaction Scheme 1 below provides one exemplary process for the preparation of chiral 1-aryl-3-azabicyclo[3.1.0]hexanes.

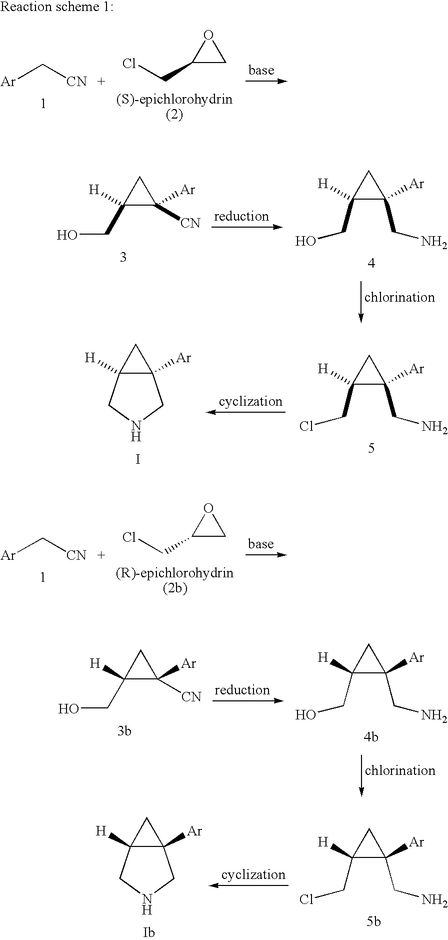

Reaction Scheme 2 below provides one exemplary process for the preparation of (1S,5R)-(−)-1-(3,4-dichlorophenyl)-3-azabicyclo[3.1.0]hexane hydrochloride. Reaction of 1-(3,4-dichlorophenyl)acetonitrile with (R)-epichlorohydiin gives approximately a 65% yield of 2-(hydroxymethyl)-1-(3,4-dichlorophenyl)cyclopropanecarbonitrile (85% cis) with the trans isomer as one of the by-products (See, Cabadio et al., Fr. Bollettino Chimico Farmaceutico 117:331-42, 1978; Mouzin et al., Synthesis 4:304-305, 1978). The cyano group of 2-(hydroxymethyl)-1-(3,4-dichlorophenyl)cyclopropanecarbonitrile can then be reduced into the amino alcohol by a reducing agent such as lithium aluminum hydride (LAH), sodium aluminum hydride (SAH) or NaBH₄ with ZnCl₂ or by catalytic hydrogenation. Cyclization of the amino alcohol with SOCl₂ or POCl₃ provides the 1-(3,4-dichlorophenyl)-3-azabicyclo[3.1.0]hexane. The cyclization of substituted 4-aminobutan-1-ol by SOCl₂ or POCl₃ into the pyrrolidine ring system has been reported previously (Armarego et al., J. Chem. Soc. [Section C: Organic] 19:3222-9, 1971; patent publication PL 120095 B2, CAN 99:158251).

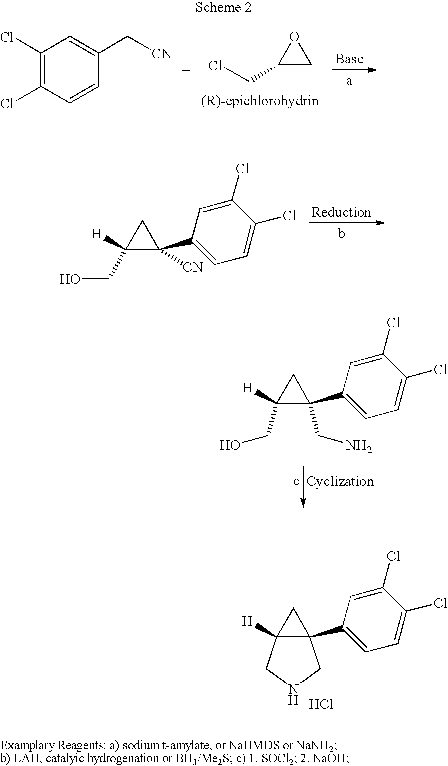

Reaction Scheme 3 below provides another exemplary process to prepare (1S,5R)-(−)-1-(3,4-dichlorophenyl)-3-azabicyclo[3.1.0]hexane hydrochloride. Reaction of 1-(3,4-dichlorophenyl)acetonitrile with (R)-epichlorohydrin gives approximately a 65% yield of 2-(hydroxymethyl)-1-(3,4-dichlorophenyl)cyclopropanecarbonitrile (85% cis) with the trans isomer as one of the by-products (See, Cabadio et al., Fr. Bollettino Chimico Farmaceutico 117:331-42, 1978; Mouzin et al., Synthesis 4:304-305, 1978). The cyano group of 2-(hydroxymethyl)-1-(3,4-dichlorophenyl)cyclopropanecarbonitrile can then be reduced into the amino alcohol by a reducing agent such as LAH, SAH or NaBH₄ with ZnCl, or by catalytic hydrogenation. The amino group can then be protected by Boc and the hydroxyl group oxidized into a carboxylic group and cyclized by treatment with TFA. Finally the lactam can be reduced into (1S,5R)-(−)-1-(3,4-dichlorophenyl)-3-azabicyclo[3.1.0]hexane by standard reducing reagents such as LAH, SAH or NaBH₄ with ZnCl₂.

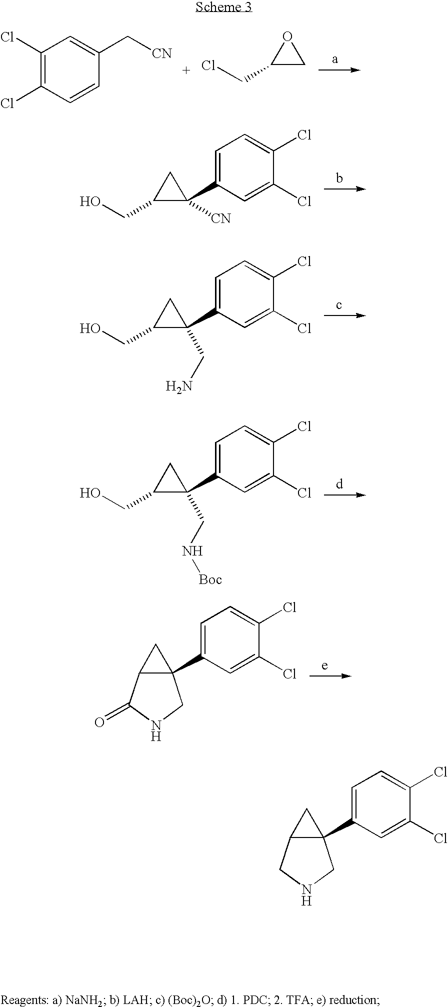

Reaction schemes 4 below provides an exemplary process to prepare chiral 1-aryl-3-azabicyclo[3.1.0]hexanes:

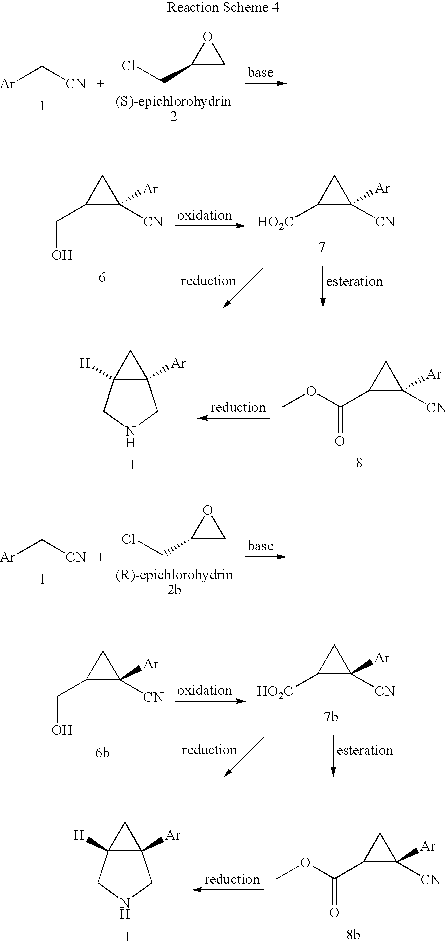

Reaction schemes 5-10 below provides additional exemplary processes to prepare 1-(3,4-dichlorophenyl)-3-azabicyclo[3.1.0]hexane, including (1S,5R)-(−)-1-(3,4-dichlorophenyl)-3-azabicyclo[3.1.0]hexane.

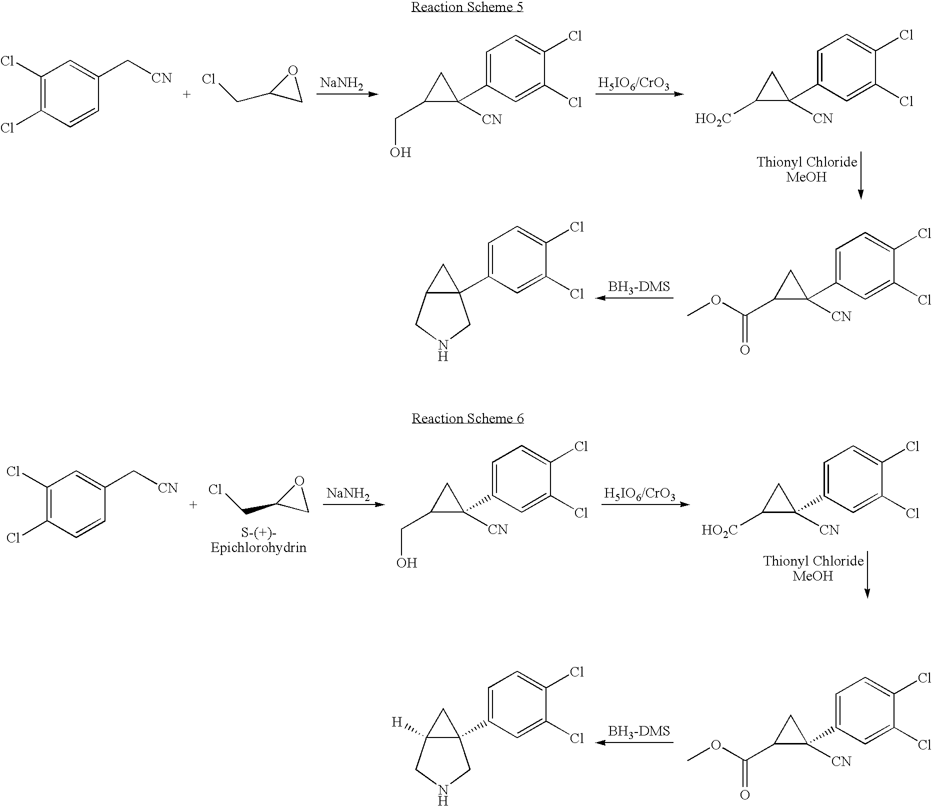

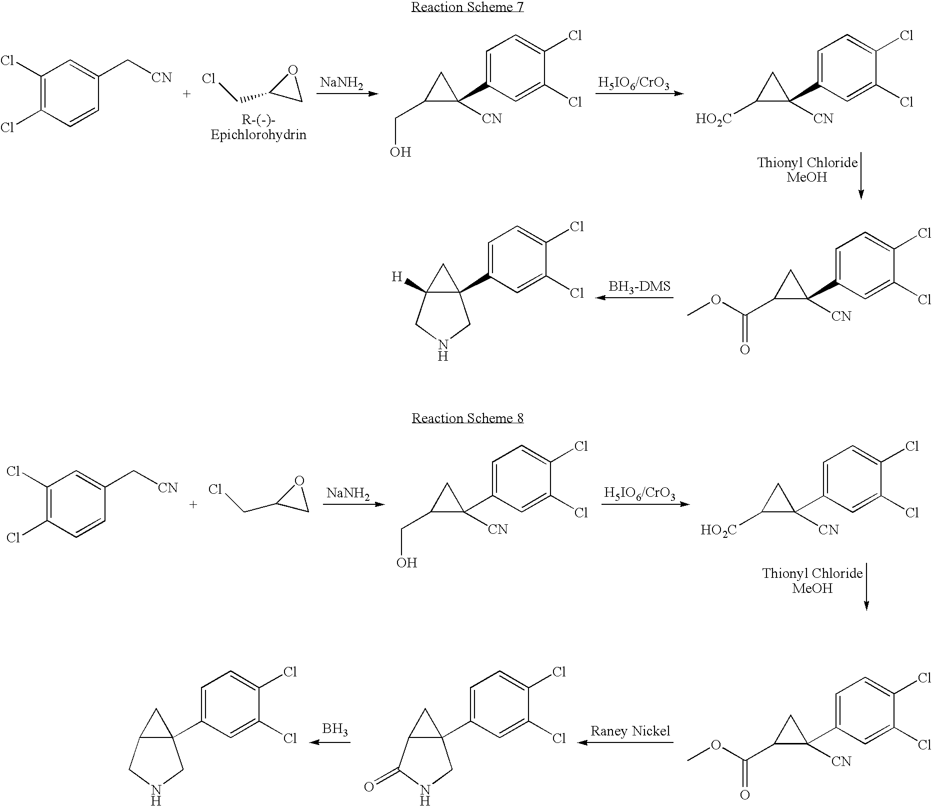

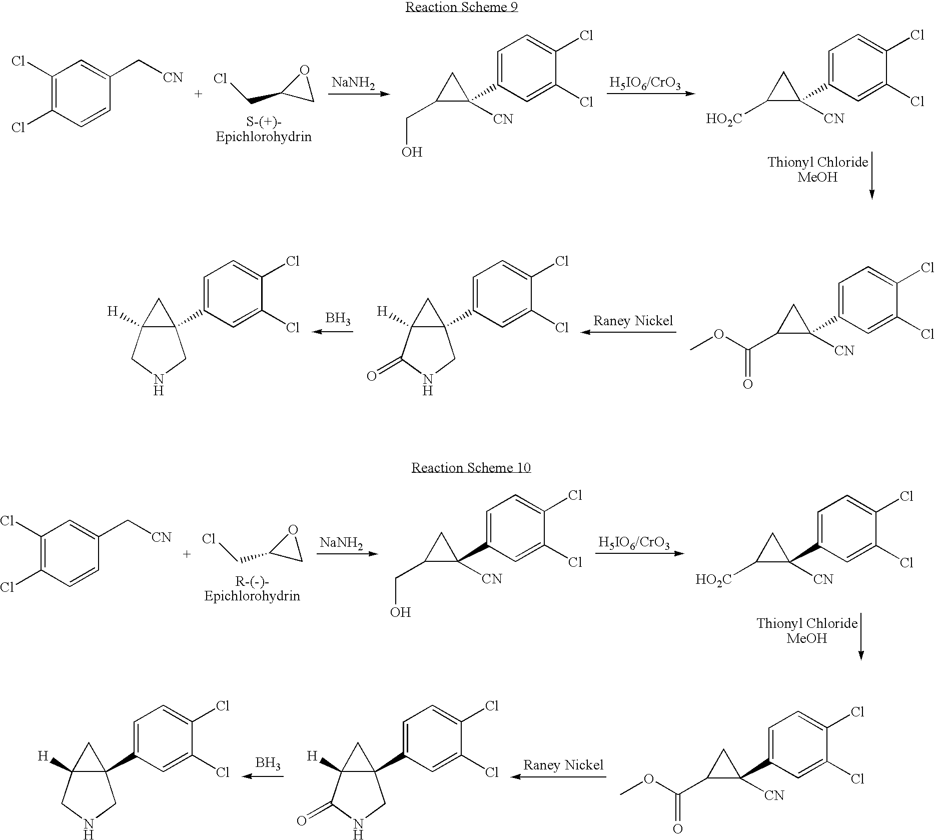

Reaction scheme 11 describe the alkylation of the chiral 1-(3,4-dichlorophenyl)-3-azabicyclo[3.1.0]hexane:

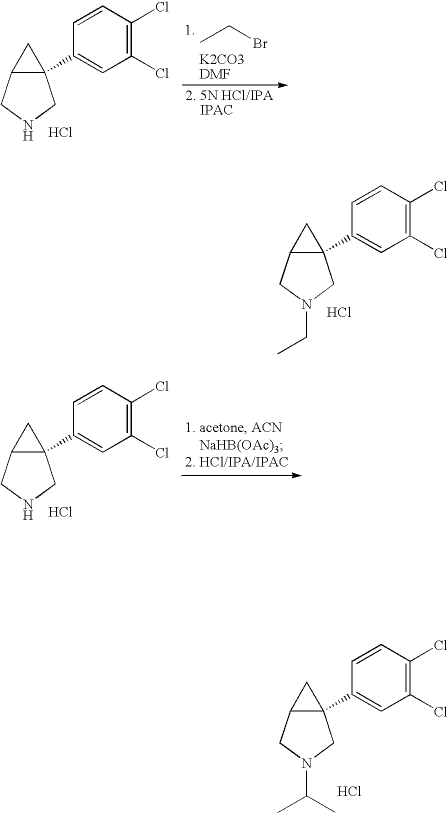

Reaction scheme 12 describe the synthesis of (1R,5S)-1-naphthalen-2-yl-3-azabicyclo[3.1.0]hexane:

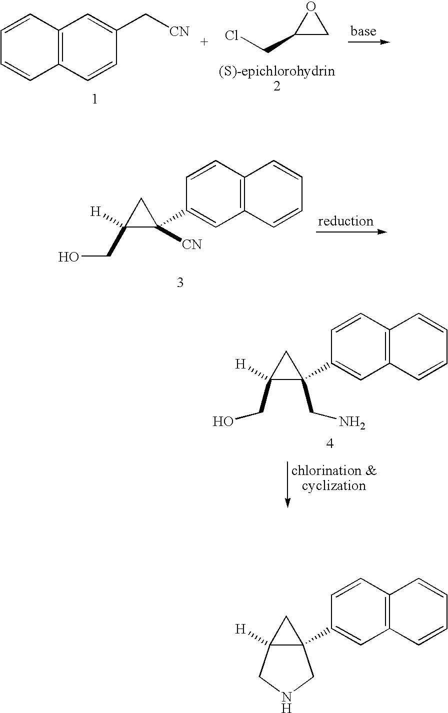

In practicing the methods of the present invention for making (1S,5R)-(−)-1-(3,4-dichlorophenyl)-3-azabicyclo[3.1.0]hexane and other 1-aryl-3-azabicyclo[3.1.0]hexanes, various reagents may be utilized for the different reaction steps. In general, suitable reagents for the various reaction steps may be selected by one of ordinary skill in the art based on the present disclosure.

In one embodiment of the present invention, for the step of contacting the starting arylacetonitrile, including for example 1-(3,4-dichlorophenyl)acetonitrile, with epichlorohydrin, or an enantiomer thereof (i.e., (S)-epichlorohydrin or (R)-epichlorohydrin) in the presence of a base to yield the cyano alcohol cyclopropyl compounds set forth above, the base that can be used includes, for example, sodium hexamethyldisilazide (NaHMDS), potassium hexamethyldisilazide (KHMDS), lithium hexamethyldisilazide (LiHMDS), potassium t-butoxide, potassium t-pentoxide, potassium amylate, sodium tert-amylate, lithium diisopropylamide (LDA), lithium tetramethylpiperidide (LiTMP), sec-butyllithium, or tert-butyllithium. Within this embodiment, the base is selected from sodium hexamethyldisilazide (NaHMDS), potassium hexamethyldisilazide (KHMDS) and lithium hexamethyldisilazide (LiHMDS). Further within this embodiment, the base is sodium hexamethyldisilazide (NaHMDS). In general, an organic solvent is used for conducting the step of contacting the arylacetonitrile, including for example 1-(3,4-dichlorophenyl)acetonitrile, with epichlorohydrin, or an enantiomer thereof (i.e., (S)-epichlorohydrin or (R)-epichlorohydrin) in the presence of a base to yield the cyano alcohol cyclopropyl compounds set forth above. Within this embodiment, suitable organic solvents include, for example, toluene, tetrahydrofuran (THF), diethyl ether, diglyme, dimethoxyethane (DME), or methyl t-butyl ether. Further within this embodiment, the organic solvent is tetrahydrofuran. The step of contacting the arylacetonitrile, including for example 1-(3,4-dichlorophenyl)acetonitrile, with epichlorohydrin, or an enantiomer thereof (i.e., (S)-epichlorohydrin or (R)-epichlorohydrin) in the presence of a base to yield the cyano alcohol cyclopropyl compounds set forth above is typically carried out at a temperature range of about −30° C. to about 25° C. Within this embodiment, the temperature is less than about 0° C. Further within this embodiment, the temperature ranges from about −20° C. to about −5° C.

In an embodiment of the present invention, for the step of reducing the cyano alcohol cyclopropyl compounds set forth above with a reducing agent to give the amino alcohol cyclopropyl compounds set forth above, suitable reducing agents include, for example, borane dimethyl sulfide complex, borane tetrahydrofuran complex, sodium borohydride-borontrifluoride etherate, dialkylboranes, 9-borabicyclo[3.3.1]nonane (9-BBN), lithium aluminum hydride (LAH), sodium aluminum hydride (SAH), NaBH₄ with ZnCl, or hydrogenation under catalyst. Further within this embodiment, the reducing agent is borane dimethyl sulfide complex. In general, an organic solvent is used for the step of reducing the cyano alcohol cyclopropyl compounds set forth above with a reducing agent to give the amino alcohol cyclopropyl compounds set forth above. Within this embodiment, suitable organic solvents include, for example, toluene, tetrahydrofuran (THF), diethyl ether, diethylene glychol dimethyl ether (diglyme), dimethoxyethane (DME), or methyl t-butyl ether. Further within this embodiment, the organic solvent is tetrahydrofuran. The step of reducing the cyano alcohol cyclopropyl compounds set forth above with a reducing agent to give the amino alcohol cyclopropyl compounds set forth above is typically carried out at a temperature range of about −30° C. to about 45° C. Within this embodiment, the temperature is less than about 0° C. Further within this embodiment, the temperature ranges from about −20° C. to about −5° C.

In an embodiment of the present invention, for the step of chlorinating the amino alcohol cyclopropyl compounds set forth above with a chlorinating agent to give the amino chloro cyclopropyl compounds set forth above, suitable chlorinating agents include, for example, thionyl chloride, SO₉Cl₂, POCl₃ and Ph₃P/CCl₄. Further within this embodiment, the chlorinating agent is thionyl chloride. In general, an organic solvent is used for conducting the step of chlorinating the amino alcohol cyclopropyl compounds set forth above with a chlorinating agent to give the amino chloro cyclopropyl compounds set forth above. Within this embodiment, suitable organic solvents include, for example, toluene, tetrahydrofuran (THF), diethyl ether, diglyme, dimethoxyethane (DME), methyl t-butyl ether, ethyl acetate, isopropyl acetate or N-methylpyrrolidinone. Further within this embodiment, the organic solvent can be tetrahydrofuran, dimethoxyethane or isopropyl acetate. The step of chlorinating the amino alcohol cyclopropyl compounds set forth above with a chlorinating agent to give the amino chloro cyclopropyl compounds set forth above is typically carried out at a temperature range of about 0° C. to about 40° C. Within this embodiment, the temperature is less than about 40° C. Further within this embodiment, the temperature is about 25° C.

In an embodiment of the present invention, for the step of cyclization of the amino chloro cyclopropyl compounds set forth above with a base to give the desired 1-aryl-3-azabicyclo[3.1.0]hexane, such as (1S,5R)-(−)-1-(3,4-dichlorophenyl)-3-azabicyclo[3.1.0]hexane, suitable bases include, for example, sodium hydroxide, ammonium hydroxide, potassium hydroxide, potassium bicarbonate, sodium bicarbonate, potassium carbonate, sodium carbonate, Et₃N, i-Pr₂NEt, DABCO, DBU, or other amine bases. Further within this embodiment, the base is sodium hydroxide. In general, an aqueous solvent is used for the step of cyclization of the amino chloro cyclopropyl compounds set forth above with a base to give the desired 1-aryl-3-azabicyclo[3.1.0]hexane, such as (1S,5R)-(−)-1-(3,4-dichlorophenyl)-3-azabicyclo[3.0]hexane. In the step of cyclodehydration of the amino chloro cyclopropyl compounds set forth above with a base to give the desired 1-aryl-3-azabicyclo[3.1.0]hexane, such as (1S,5R)-(−)-1-(3,4-dichlorophenyl)-3-azabicyclo[3.1.0]hexane, the pH typically ranges of from about 7 to about 10. Within this embodiment, the pH ranges from about 8 to about 10. Further within this embodiment, the pH ranges from about 8.5 to about 9.5.

Suitable nitrogen protecting groups that can be used in the methods of the present invention include, for example, benzyl, allyl, tert-butyl and 3,4-dimethoxy-benzyl groups. In general, nitrogen protecting groups are well known to those skilled in the art, see for example, “Nitrogen Protecting Groups in Organic Synthesis”, John Wiley and Sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry”, 3rd edition, John Wiley & Sons, New York, N.Y., 1999.

When the nitrogen protecting group is no longer needed, it may be removed by methods well known in the art. For example, benzyl or 3,4-dimethoxy-benzyl groups may be removed by catalytic hydrogenation. In general, methods of removing nitrogen protecting groups are well known to those skilled in the art, see for example, “Nitrogen Protecting Groups in Organic Synthesis”, John Wiley and Sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry”, 3rd edition, John Wiley & Sons, New York, N.Y., 1999.

In performing the methods of the present invention, other suitable reagents for causing halogenation and cyclization include, for example, SOCl₂, POCl₃, oxalyl chloride, phosphorous tribromide, triphenylphosphorous dibromide and oxalyl bromide.

The present invention also provides useful compounds for the production of (1S,5R)-(−)-1-(3,4-dichlorophenyl)-3-azabicyclo[3.1.0]hexane and other chiral 1-aryl-3-azabicyclo[3.1.0]hexanes.

In certain embodiments, the present invention provides a compound selected from the group consisting of compounds having the following formulas:

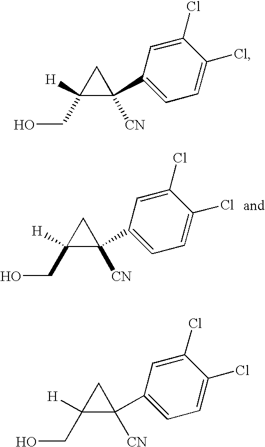

In additional embodiments, the present invention provides a compound selected firm the group consisting of compounds having the following formulas and pharmaceutically acceptable salts thereof:

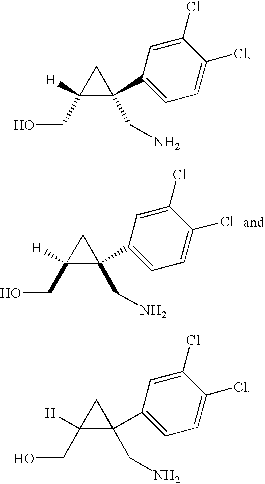

The present invention further provides useful compounds for the production of (1R,5S)-1-naphthalen-2-yl-3-azabicyclo[3.1.0]hexane.

In certain embodiments, the present invention provides a compound selected from the group consisting of compounds having the following formulas: 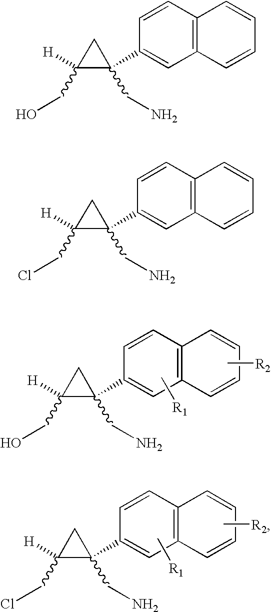
wherein R₁ and R₂ are independently selected from hydrogen, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

In other embodiments, the present invention provides a compound selected from the group consisting of compounds having the following formulas: 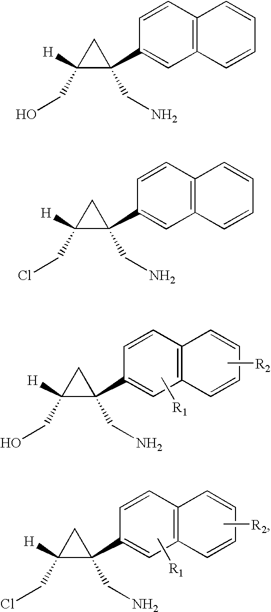
wherein R₁ and R₂ are independently selected from hydrogen, halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

In additional embodiments, the present invention provides a compound selected from the group consisting of compounds having the following formulas: 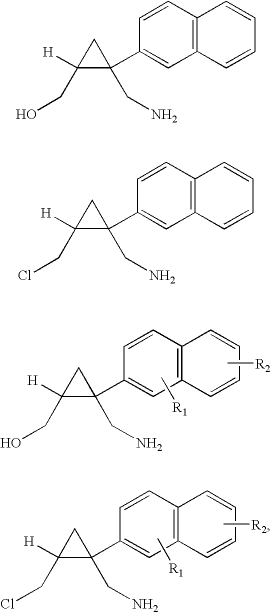
wherein R₁ and R₂ are independently selected from hydrogen, halogen, C₁₋₃ alkyl, C₁₋₄ alkenyl, C₂)₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.

For the purposes of describing the present invention, the following terms and definitions are provided by way of example. Additional terms and definitions for describing the present invention are provided by way of example elsewhere in the application.

As used herein, the term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction.

The term “racemic” refers to a mixture of equal parts of enantiomer and which is optically inactive.

The term “resolution” refers to the separation or concentration or depletion of one of the two enantiomeric forms of a molecule.

Suitable forms of (−)-1-(3,4-dichlorophenyl)-3-azabicyclo[3.1.0]hexane and other 1-aryl-3-azabicyclo[3.1.0]hexanes for use in biologically active compositions and methods of the present invention include their pharmaceutically acceptable salts, polymorphs, solvates, hydrates, and prodrugs.

As noted above, the methods of the present invention can be used to prepare pharmaceutically acceptable salts of (−)-1-(3,4-dichlorophenyl)-3-azabicyclo[3.1.0]hexane and other 1-aryl-3-azabicyclo[3.1.0]hexanes, including both acid addition salts formed from an acid and the basic nitrogen group of (−)-1-(3,4-dichlorophenyl)-3-azabicyclo[3.1.0]hexane and other 1-aryl-3-azabicyclo[3.1.0]hexanes and base salts. Suitable acid addition salts include, for example, hydrochloride, hydrobromide, hydroiodide, sulphate, hydrogen sulphate, nitrate, phosphate, and hydrogen phosphate salts. Other examples of pharmaceutically acceptable acid addition salts include inorganic and organic acid addition salts. Additional pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. Suitable base salts are formed from bases, which form non-toxic salts and include, for example, aluminum, calcium, lithium, magnesium, potassium, sodium, zinc and diethanolamine salts. The hydrochloride salt formed with hydrochloric acid is an exemplary useful salt.

Compounds produced in accordance with the methods of the invention may be utilized in treating or preventing a wide variety of central nervous system (CNS) disorders in mammals. Mammalian subjects amenable for treatment according to the methods of the invention include, but are not limited to, human and other mammalian subjects suffering from a CNS disorder, including a neurological or psychiatric condition, that is amenable to treatment or beneficial intervention using a compound produced in accordance with the methods of the present invention. Disorders for which the compounds produced in accordance with the methods of the present invention may be useful include irritable bowel syndrome; inflammatory bowel disease; bulimia; anorexia; obesity and related eating disorders; urinary tract disorders, such as stress urinary incontinence; addictive disorders (including addiction to nicotine, stimulants, alcohol, and opiates); degenerative diseases, including Alzheimer's disease, amyotrophic lateral sclerosis, and Parkinson's disease; and pyretic conditions (including fevers, and post- and peri-menopausal hot flashes).

Administration of an effective amount of a compound produced in accordance with the methods of the present invention to a mammalian subject presenting with a CNS disorder, including a neurological or psychiatric condition in the subject, will detectably treat, alleviate, eliminate, or prevent the targeted CNS disorder and/or one or more symptom(s) associated therewith. In exemplary embodiments, administration of a compound produced in accordance with the methods of the present invention to a suitable test subject will yield a reduction in the targeted CNS disorder, or one or more targeted symptom(s) associated therewith, such as depression, by at least 10%, 20%, 30%, 50% or greater, up to a 75-90%, or 95% or greater, reduction in the one or more target symptom(s), compared to placebo-treated or other suitable control subjects. Comparable levels of efficacy are contemplated for the entire range of CNS disorders described herein, including all contemplated neurological and psychiatric disorders, as well as all other CNS conditions and symptoms identified herein for treatment or prevention using the compounds produced in accordance with the methods of the present invention.

It should be understood that a compound produced in accordance with the methods of the invention can be combinatorially formulated or coordinately administered with a second therapeutic agent or method—yielding an effective formulation or method to alleviate or prevent a CNS disorder and/or one or more symptom(s) associated therewith in a mammalian subject. Accordingly, a compound produced in accordance with the methods of the invention may be utilized in combinatorial formulations and coordinate administration methods which employ an effective amount of a compound produced in accordance with the methods of the invention, and one or more additional active agent(s) that is/are combinatorially formulated or coordinately administered with such compound to yield a combinatorial formulation or coordinate administration method that is effective to alleviate or prevent a CNS disorder and/or one or more symptom(s) associated therewith in a mammalian subject.

Additionally, a compound produced in accordance with the methods of the invention (or a pharmaceutically effective salt, solvate, hydrate, polymorph, or prodrug thereof) can be used in combination therapy with at least one other therapeutic agent or method. In this context, a compound produced in accordance with the methods of the invention (or a pharmaceutically effective salt, solvate, hydrate, polymorph, or prodrug thereof) can be administered concurrently or sequentially with administration of a second therapeutic agent, for example a second agent that acts to prevent or treat the same or a different disorder or symptom(s) from which the compound produced in accordance with the methods of the invention (or a pharmaceutically effective salt, solvate, hydrate, polymorph, or prodrug thereof) is administered. The compound produced in accordance with the methods of the invention (or a pharmaceutically effective salt, solvate, hydrate, polymorph, or prodrug thereof) and the second therapeutic agent can be combined in a single composition or administered in different compositions. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the compound produced in accordance with the methods of the invention (or a pharmaceutically effective salt, solvate, hydrate, polymorph, or prodrug thereof) exerts at least some detectable therapeutic activity towards alleviating or preventing a CNS disorder and/or one or more symptom(s) associated therewith in a mammalian subject, which may or may not be in conjunction with a secondary clinical response provided by the secondary therapeutic agent. Often, the coordinate administration of a compound produced in accordance with the methods of the invention (or a pharmaceutically effective salt, solvate, hydrate, polymorph, or prodrug thereof) with a secondary therapeutic agent as contemplated herein will yield an enhanced therapeutic response beyond the therapeutic response elicited by either or both the compound produced in accordance with the methods of the invention (or a pharmaceutically effective salt, solvate, hydrate, polymorph, or prodrug thereof) and/or secondary therapeutic agent alone.

Since a compound produced in accordance with the methods of the invention may need to be administered to a patient chronically for the purpose of preventing or treating a particular CNS disorder, in one embodiment combination therapy involves alternating between administering a compound produced in accordance with the methods of the invention (or a pharmaceutically effective salt, solvate, hydrate, polymorph, or prodrug thereof) and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

The active compounds produced in accordance with the methods of the invention may be optionally formulated with a pharmaceutically acceptable carrier and/or various excipients, vehicles, stabilizers, buffers, preservatives, etc. An “effective amount,” “therapeutic amount,” “therapeutic effective amount,” or “effective dose” is an effective amount or dose of an active compound as described herein sufficient to elicit a desired pharmacological or therapeutic effect in a mammalian subject—typically resulting in a measurable reduction in an occurrence, frequency, or severity of one or more symptom(s) associated with or caused by a CNS disorder, including any combination of neurological or psychological symptoms, diseases, conditions, or disorders associated with or caused by the targeted CNS disorder, in the subject. In certain embodiments, when a compound produced in accordance with the methods of the invention is administered to treat a CNS disorder, for example depression, an effective amount of the compound will be an amount sufficient in vivo to delay or eliminate onset of symptoms of the targeted condition or disorder. Therapeutic efficacy can alternatively be demonstrated by decrease in the frequency or severity of symptoms associated with the treated condition or disorder, or by altering the nature, recurrence, or duration of symptoms associated with the treated condition or disorder. Therapeutically effective amounts, and dosage regimens, of the compounds produced in accordance with the methods of the invention, including pharmaceutically effective salts, solvates, hydrates, polymorphs or prodrugs thereof, will be readily determinable by those of ordinary skill in the art, often based on routine clinical or patient-specific factors.

Suitable routes of administration for a compound produced in accordance with the methods of the present invention include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a compound produced in accordance with the methods of the invention for mammalian subjects may range from about 1 to 1200 mg, 50 to 1000 mg, 75 to 900 mg, 100 to 800 mg, or 150 to 600 mg. In certain embodiments, the effective unit dosage will be selected within narrower ranges of, for example, 10 to 25 mg, 30 to 50 mg, 75 to 100 mg, 100 to 150 mg, 150 to 250 mg or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of 10 to 25 mg, 30 to 50 mg, 75 to 100 mg, 100 to 200 (anticipated dosage strength) mg, or 250 to 500 mg, are administered one, two, three, or four times per day. In more detailed embodiments, dosages of 50-75 mg, 100-150 mg, 150-200 mg, 250-400 mg, or 400-600 mg are administered once, twice daily or three times daily. In alternate embodiments, dosages are calculated based on body weight, and may be administered, for example, in amounts from about 0.5 mg/kg to about 30 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions comprising an effective amount of a compound produced in accordance with the methods of the present invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for a compound produced in accordance with the methods of the invention will ordinarily be selected to approximate a minimal dosing regimen that is necessary and sufficient to substantially prevent or alleviate one or more symptom(s) of a CNS disorder in the subject, as described herein. Thus, following administration of a compound produced in accordance with the methods of the present invention, test subjects will exhibit a 10%, 20%, 30%, 50% or greater reduction, up to a 75-90%, or 95% or greater, reduction, in one or more symptoms associated with a targeted CNS disorder, including any targeted neurological or psychiatric disorder, such as depression, compared to placebo-treated or other suitable control subjects.

Pharmaceutical dosage forms of a compound produced in accordance with the methods of the present invention may optionally include excipients recognized in the art of pharmaceutical compounding as being suitable for the preparation of dosage units as discussed above. Such excipients include, without limitation, binders, fillers, lubricants, emulsifiers, suspending agents, sweeteners, flavorings, preservatives, buffers, wetting agents, disintegrants, effervescent agents and other conventional excipients and additives.

The compositions comprising an effective amount of a compound produced in accordance with the methods of the present invention for treating CNS disorders can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

If desired, a compound produced in accordance with the methods of the present invention can be administered in a controlled release form by, for example, use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps.

A compound produced in accordance with the methods of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms include tablets, which may be prepared by any conventional method of preparing pharmaceutical oral unit dosage forms. Oral unit dosage forms, such as tablets, may contain one or more conventional additional formulation ingredients, including, but not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and in certain embodiments in less than thirty seconds. By effervescent agent is meant a couple, typically an organic acid and a carbonate or bicarbonate. Such rapidly acting dosage forms would be useful, for example, in the prevention or treatment of acute attacks of panic disorder.

The compositions comprising an effective amount of a compound produced in accordance with the methods of the present invention can be prepared and administered in any of a variety of inhalation or nasal delivery forms known in the art. Devices capable of depositing aerosolized formulations of a compound produced in accordance with the methods of the present invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound produced in accordance with the methods of the present invention, and any additional active or inactive ingredient(s).

Intranasal and pulmonary delivery permits the passage of active compounds to the blood stream directly after administering an effective amount of the compound to the nose or lung. In the case of intranasal delivery, this mode of delivery can achieve direct, or enhanced, delivery of an active compound to the CNS.

For intranasal and pulmonary administration, a liquid aerosol formulation will often contain an active compound produced in accordance with the methods of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle of a sufficiently small particle diameter, e.g., in a range of from about 2-5 microns, suitable for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Compounds produced in accordance with the methods of the invention may also be utilized in topical administration methods and compositions for treating CNS disorders. Topical compositions may comprise a compound produced in accordance with the methods of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound produced in accordance with the methods of the present invention dissolved or dispersed in a portion of water or other solvent or liquid to be incorporated in the topical composition or delivery device. It can be readily appreciated that the transdermal route of administration may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound produced in accordance with the methods of the present invention are provided for parenteral administration (e.g., intravenously, intramuscularly, subcutaneously or intraperitoneally), including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The parenteral preparations may be solutions, dispersions or emulsions suitable for such administration. Pharmaceutically acceptable formulations and ingredients will typically be sterile or readily sterilizable, biologically inert, and easily administered. Parenteral preparations typically contain buffering agents and preservatives, and may be lyophilized to be re-constituted at the time of administration. The formulations may be presented in unit-dose or multi-dose containers.

Formulations comprising an effective amount of a compound produced in accordance with the methods of the present invention may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Exemplary unit dosage formulations contain a daily dose or unit, daily sub-dose, as described herein above, or an appropriate fraction thereof, of the active ingredient(s).

A compound produced in accordance with the methods of the present invention may be encapsulated for delivery in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

The following examples illustrate certain embodiments of the present invention, and are not to be construed as limiting the present disclosure.

### EXAMPLE I

### A. Preparation of (1S,2R)-1-(3,4-dichlorophenyl)-2-hydroxymethyl-cyclopropanecarbonitrile

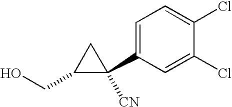

A 1000 mL triple neck flask was charged with dry THF (300 mL) and (3,4-dichlorophenyl)-acetonitrile (30 g, 161 mmol). The reaction was cooled to −25° C. and sodium amide (6.3 g, 161 mmol, 1 eq) was added in one portion. The reaction was allowed to warm to 20° C. over two hours. The reaction was than cooled to −25° C. and R-(−)-epichlorohydrin (14.8 g, 161 mmol) was added followed by sodium amide (6.3 g, 161 mmol, 1 eq). The reaction was slowly allowed to warm to 15° C. over 8 hrs. The reaction mixture was slowly added to saturated ammonia chloride (750 mL) with stirring and ethyl acetate (1 L) was then added. The organic layer was separated, dried with magnesium sulfate and reduced to a dark yellow oil. Purification via the ISCO Companion system (120 gram column) eluting with 1-5% methanol in dichloromethane affords 24 g, 61% yield (3.6:1 E/Z) of a yellow oil. ¹H NMR (400 MHz, CDCl₃-d partial assignment) δ ppm 1.57-1.72 (m, 3H) 1.88-1.99 (m, 1H) 3.81 (dd, J=12.10, 8.20 Hz, 1H) 4.13 (dd, J=12.10, 5.08 Hz, 1H) 7.17 (dd, J=8.40, 2.34 Hz, 1H) 7.40 (d, J=2.34 Hz, 1H) 7.44 (d, J=8.40 Hz, 1H)

### B. Preparation of ((1R,2S)-2-(aminomethyl)-2-(3,4-dichlorophenyl)cyclopropyl)methanol

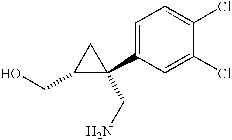

A 1000 mL triple neck flask was charged with THF (150 mL) and cooled to 0° C. Lithium aluminum hydride (13 grams, 0.350 mol) was added slowly. A solution of 1-(3,4-dichloro-phenyl)-2-hydroxymethyl-cyclopropanecarbonitrile (24 g, 0.099 mol) in THF (150 mL) was added dropwise, keeping the reaction temperature below 5° C. The reaction was stirred between 0° C. and 5° C. for 3 hours, at which time the reaction was complete by HPLC. Once the reaction was complete, sodium sulfate decahydrate (250 grams) was slowly added keeping the temperature below 10° C. Once the exotherm subsides the reaction was stirred for 16 hours. The reaction was then filtered, and the filter cake washed with THF (300 mL). The filtrate was concentrated to afford an oil which was purified using the ISCO companion purification system (120 gram column). Fractions are collected and concentrated to afford 11 grams (45%) of an oil. ¹H NMR (400 MHz, CHLOROFORM-d) δ ppm 0.75-0.80 (m, 1H) 0.95 (dd, J=8.79, 4.88 Hz, 1H) 1.65-1.77 (m, 1H) 2.59 (d, J=12.69 Hz, 1H) 3.33 (dd, J=12.30, 10.93 Hz, 1H) 3.43 (dd, J=12.89, 0.98 Hz, 1H) 4.11 (dd, J=12.30, 5.47 Hz, 1H) 7.24 (dd, J=8.30, 2.05 Hz, 1H) 7.39 (d, J=8.20 Hz, 1H) 7.49 (d, J=2.15 Hz, 1H)

### C. Preparation of tert-butyl ((1S,2R)-1-(3,4-dichlorophenyl)-2-(hydroxymethyl)-cyclopropyl)methylcarbamate

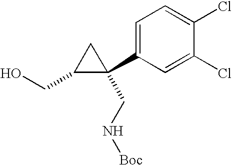

A solution of [2-Aminomethyl-2-(3,4-dichloro-phenyl)-cyclopropyl]-methanol (11.0 g, 44 mmol) and di-tert-1-butyl-dicarbonate (Boc₂O, 11.0 g, 49 mmol) in dichloromethane (200 mL) was stirred at room temperature for 16 hours. To the reaction, water (200 mL) was added, and the resulting mixture was partitioned. The organic layer was separated, washed with brine and dried with magnesium sulfate. The dichloromethane was removed under reduced pressure to yield the product as an oil, 15.2 g (95%). This was used for the preparation of (1S,2R)-(−)1-(3,4-Dichloro-phenyl)-4-oxo-3-aza-bicyclo[3.1.0]hexane-3-carboxylic acid tert-butyl ester without further purification.

### D. Preparation of (1S,2R)-(−)1-(3,4-Dichlorophenyl)-4-oxo-3-azabicyclo[3.1.0]hexane-3-carboxylic acid tert-butyl ester

A mixture of [1-(3,4-Dichlorophenyl)-2-hydroxymethyl-cyclopropylmethyl]-carbamic acid tert-butyl ester (15.2 g, 44 mmol), PDC (33 g, 88 mmol) and molecular sieves 4 A (powder, 20 g) in dichloromethane (300 mL) was stirred at room temperature for 4 hours. To the reaction, diethyl ether (50 mL) was added and the resulting mixture was filtered through celite and the filtrate was evaporated. The residue was purified via the ISCO Companion chromatography eluting with 1-2% methanol in dichloromethane to afford 2.0 g (13%) of a white solid. ¹H NMR (400 MHz, CHLOROFORM-d) δ ppm 1.33 (dd, J=4.98, 3.61 Hz, 1H) 1.52 (s, 9H) 1.58 (dd, J=9.08, 4.98 Hz, 1H) 2.26-2.31 (m, 1H) 3.88-3.95 (m, 5H) 4.00-4.05 (m, 1H) 7.09 (dd, J=8.40, 2.15 Hz, 1H) 7.36 (d, J=2.15 Hz, 1H) 7.43 (d, J=8.20 Hz, 1H)

### E. Preparation of (1S,5R)-(−)-5-(3,4-Dichlorophenyl)-3-azabicyclo[3.1.0]hexan-2-one

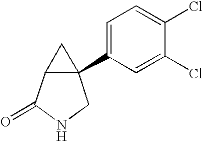

To a solution of 1-(3,4-dichlorophenyl)-4-oxo-3-aza-bicyclo[3.1.0]hexane-3-carboxylic acid tert-butyl ester (2.0 g, 5 mmol) in dichloromethane (200 mL) at 0° C. was added trifluoroacetic acid (15 mL) dropwise over a period of 15 minutes. The reaction was allowed to warm to room temperature and stir for 6 hours, at which time the reaction was complete by TLC. After the solvent was evaporated, the residue was dissolved in dichloromethane (200 mL) and washed with saturated sodium bicarbonate (250 mL), water (200 mL), and brine (200 mL) and dried with magnesium sulfate. The organic layer was reduced to a white solid. Pure product was obtained 300 mg (22%) after recrystallization from chloroform. MS [M+1] 243, Purity 99.0% AP, 99.5% ee. Optical rotation −35.6 @ 25° C., concentration 0.972 g/100 mL (methanol). ¹H NMR (400 MHz, DMSO-d₆) δ ppm 1.08 (t, J=4.00 Hz, 1H) 1.44 (dd, J=8.93, 4.39 Hz, 1H) 2.19-2.26 (m, 1H) 3.57 (d, J=3.03 Hz, 2H) 7.25 (dd, J=8.40, 2.20 Hz, 1H) 7.37 (s, 1H) 7.53 (d, J=2.20 Hz, 1H) 7.56 (d, J=8.40 Hz, 1H). ¹³C NMR (101 MHz, DMSO-d₆) δ ppm 20.50 (s, 1C) 27.18 (s, 1C) 28.59 (s, 1C) 46.89 (s, 1C) 126.83 (s, 1C) 128.53 (s, 1C) 130.24 (s, 1C) 130.96 (s, 1C) 141.56 (s, 1C) 175.40 (s, 1C)

### EXAMPLE II

### Preparation of (1S,5R)-(−)-1-(3,4-dichlorophenyl)-3-azabicyclo[3.1.0]hexane HCl salt

### A. Preparation of (1S,2R)-1-(3,4-Dichlorophenyl)-2-hydroxymethyl-cyclopropane carbonitrile

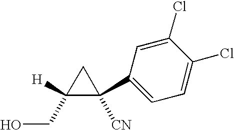

Apparatus:

750 ml four-necked flask, ancer stirrer, addition funnel, inside thermometer, cooling bath, inert gas (Argon)

Chemicals:

(3,4-dichlorophenyl)acetonitrile (DPA): 97%, Fluka 35530

(R)-epichlorohydrin: 99.0%, 99.2% ee, Rhodia Pharma solutions Batch 700019280, d=1.18

sodium tert-amylate: 95%, Aldrich 280704

Tetrahydrofurane (THF): Synopharm/Schweizerhall, d=0.889

Tert-butyl methylether (TBME): Synopharm/Schweizerhall, d=0.74

Toluene: Synopharm/Schweizerhall, d=0.865

Operation Mode:

The flask was charged with 45.8 g (395 mmol, 2.10 equiv) sodium tert-amylate, solubilized in 400 ml Tetrahydrofurane and cooled to −5° C. (inside temperature). On cooling the base partially precipitated affording a white suspension that was still well stirrable. A solution of 35.0 g (188 mmol, 1 equiv) (3,4-dichlorophenyl)acetonitrile in 150 ml Tetrahydrofurane was added to the suspension within 5 min under stirring using a dropping funnel. During addition the inside temperature was kept between −5 and −3° C. The original suspension turned to a yellowish solution. After stirring another 5 min, 16.3 ml (207 mmol, 1.1 equiv) (R)-epichlorohydrin were added to the mixture within 15 min keeping the temperature at −3° C. (1° C.). The now red-orange solution was stirred at 0° C. After 2 h another 0.74 ml (9.4 mmol, 0.05 equiv) (R)-epichlorohydrin were added for full conversion (HPLC-control). After another 2.5 h at 0° C. the mixture was cooled to −58° C. At this temperature 62.7 ml (250 mmol, 1.3 equiv) of a 4 M solution of HCl in dioxane were added within 7 min keeping the inside temperature between −58° C. and −50° C. (as the HCl quench is exothermic, the addition of the acid solution to the basic medium has to be carefully controlled). The red-orange solution turns to an orange suspension. The cooling bath was removed and the mixture allowed warming to 0° C.

Work-Up:

The still cold mixture was taken in a tert-butyl methyl ether (11) washed three (3) times with water (500 ml each). The combined water layer was reextracted once with tert-butyl methyl ether (11). The combined organic layer was dried over sodium sulphate, filtered and concentrated on rotavap (20 mbar, bath temperature: 50° C.). The residue was stripped twice with toluene and dried (HV, 50° C., 16 h) affording 45.8 g (100% crude yield) of a red-orange crude oil.

The NMR and HPLC spectra of the crude material show a ˜5.3:1 ratio of Z to E-isomer. The HPLC purity of Z+E was ˜89 area %@220 nm. ˜8% of lactone side product are in the crude material and ˜1% of iminoether.

Analysis:

¹H NMR (CDCl₃, 300 MHz) δ 7.45 (d, J=8.4 Hz, 1H), 7.40 (d, J=2.4 Hz, 1H), 7.16 (dd, J=8.4, 2.4 Hz, 1H), 4.10 (dd, J=12.0, 5.1 Hz, 1H), 3.75 (dd, J=12.0, 8.2 Hz, 1H), 1.96-1.87 (m, 1H, ArCCH2CH), 1.67-1.42 (m, 2H, ArCCH2CH). (Z-isomer)

HPLC: Rt=7.56 min (Z-isomer); 7.39 min (E-isomer)

### B. Preparation of [2-Aminomethyl-2-(3,4-dichlorophenyl)-cyclopropyl]-methanol HCl salt

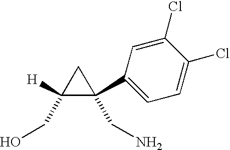

Apparatus:

1 l autoclave

Chemicals:

Methanol: Synopharm/Schweizerhall, d=0.791

Ammonia (g):

RaCo SK03/06; Grace-Raney 2724

Acetonitrile: Synopharm/Schweizerhall, d=0.786

HCl, 5-6M in 2 PrOH, Acros 133700010, d=0.909

Diethyl ether: Synopharm/Schweizerhall, d=0.706

Operation Mode:

The autoclave was charged with 400 ml methanol and 40.0 g (mmol) of crude (1S,2R)-1-(3,4-Dichlorophenyl)-2-hydroxymethyl-cyclopropane carbonitrile and 40.0 g of RaCo SK03/06 (prewashed with methanol). The autoclave was closed and purged three times with nitrogen (5 bar), then four times with hydrogen (5 bar). Then 60 g of ammonia were charged in the autoclave. The heating and stirring were switched on and when the inside temperature reached 80° C.; the pressure was set to 50 bar (725 psi). After 6.5 h, the autoclave was cooled to room temperature and the pressure was released. ˜6.41 hydrogen were consumed, i.e. 87% of the theoretical consumption. HPLC analysis of an aliquot showed the reaction to be finished.

Work-Up:

The autoclave was purged with nitrogen (4×5 l) and the mixture filtered over a pressure filter system using a Hyflo pad within 10 min. The Hyflo pad was washed with 100 ml methanol and the combined filtrate was concentrated under reduced pressure on rotavap (20 mbar, bath temperature 45° C.) to afford 40.2 g of a yellow-brownish crude oil. The crude oil was partitioned between 150 ml aqueous 2 M HCl and 100 ml dichloromethane. The aqueous layer was washed with dichloromethane (1×60 ml, 1×30 ml) to remove non-basic impurities. The aqueous layer was brought to pH=8 with ammonium hydroxide and reextracted with dichloromethane (3×100 ml). These latter organic layers were combined, dried over sodium sulphate, filtered and evaporated to afford 32.5 g of yellowish oil.

Purification:

The material was dissolved in 45 ml acetonitrile and 1 equiv HCl (5-6 M in 2-propanol) were added using an ice-cooling bath. ˜130 ml of diethyl ether were added. After ˜10 minutes, spontaneous crystallization occurred. The crystals were allowed to settle for 3 days and the crystals were filtered, washed with 2×50 ml diethyl ether/acetonitrile (1:1) and diethyl ether (1×50 ml) and dried in vacuum (20 mbar, 60° C., 2 h) to afford 16.9 g (36% yield) of the title compound HCl salt as white crystals. HPLC spectra of the crystals showed a −94% chemical purity of desired Z-isomer. ˜6% of E-isomer+mono-chlorophenyl impurity was present in the crystals.

Analysis:

¹H-NMR

Free Base:

¹H NMR (CDCl₃, 300 MHz) δ 7.50 (d, J=2.0 Hz, 1H), 7.39 (d, J=8.3 Hz, 1H), 7.24 (dd, J=8.3, 2.0 Hz, 1H), 4.12 (dd, J=12.3, 5.4 Hz, 1H), 3.46-3.30 (m, 2H), 2.60 (d, J=12.8 Hz, 1H), 2.75-2.68 (m, 1H, ArCCH2CH), 0.95 (dd, J=8.7, 5.1 Hz, 1H, ArCCH2CH), 0.80-0.76 (m, 1H, ArCCH2CH).

HCl salt:

¹H NMR (d6-DMSO, 300 MHz) δ 7.98 (bs, 3H, NH3Cl), 7.67 (dd, J=4.8, 2.1 Hz, 1H), 7.60 (d, J=8.4 Hz, 1H), 7.39 (dd, J=8.4, 4.8 Hz, 1H), 5.23 (bs, 1H, OH), 3.86 (dd, J=12.0, 5.1 Hz, 1H), 1.48-1.38 (m, 1H, ArCCH2CH), 1.15 (dd, J=8.7, 5.1 Hz, 1H, ArCCH2CH), 1.03-0.99 (m, 1H, ArCCH2CH).

¹³C-NMR(HCl Salt) 143.63; 131.86; 131.27; 130.91; 130.02; 129.92; 60.20; 43.39; 28.04; 27.49; 16.81.

HPLC: Rt=5.72 min

MS: m/z=246 [M]+

### C. Preparation of (1S,5R)-(−)-1-(3,4-dichlorophenyl)-3-azabicyclo[3.1.0]hexane HCl salt

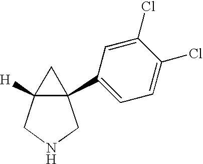

Apparatus:

750 ml four-necked flask, ancre stirrer, addition funnel, inside thermometer, cooling bath, inert gas (Argon)

Chemicals:

Thionyl chloride: 99%, Fluka 88950, d=1.635

NH4OH: 25% Fluka 09860; d=0.91

Ethyl acetate: Synopharm/Schweizerhall, d=0.902

2-Propanol: Synopharm/Schweizerhall, d=0.785

37% conc. aq. HCl: Fluka 84426, d=1.18

Diethyl ether: Synopharm/Schweizerhall, d=0.706

Operation Mode:

The flask was charged with 400 ml ethyl acetate. 16.0 g (56.6 mmol, 1 equiv) of [2-aminomethyl-2-(3,4-dichlorophenyl)-cyclopropyl]-methanol HCl salt were added and the stirred white suspension was cooled to 0° C. (inside temperature). At this temperature 5.3 ml (73.6 mmol, 1.3 equiv) of thionyl chloride were added dropwise via addition funnel within 5 min. After full addition the mixture was stirred 2 h at low temperature (−2 to 0° C.). The initial white suspension turned almost completely homogeneous and turned then again into a suspension of the intermediate chloro ammonium salt (HPLC control of an aliquot showed the chlorination step to be complete). The mixture was cooled to −10° C. then 33.8 ml (453 mmol, 8.0 equiv) of 25% ammonium hydroxide were added within 30 min keeping the inside temperature between −10 and −5° C. The now colorless emulsion was stirred at 0° C. for 2 h then at room temperature for 2 h. (HPLC control of an aliquot showed the cyclization to be finished).

Work-Up:

The reaction mixture was diluted with water and ethyl acetate. The aqueous layer was separated and extracted once with ethyl acetate. The combined ethyl acetate layer was washed with water, dried over sodium sulphate and concentrated on rotavap (20 mbar, bath temperature 45° C.) to afford 12.9 g of clear golden oil.

Purification:

The crude material was dissolved in 20 ml 2-propanol under stirring. 1 equiv of HCl (5-6 M in 2-Propanol) was added. A spontaneous crystallization occurred. After additional 10 min stirring the white crystals were filtered and washed with diethyl ether to afford 11.8 g (79%) of (1S,5R)-(−)-1-(3,4-dichlorophenyl)-3-azabicyclo[3.1.0]hexane HCl. The NMR and HPLC spectra of the crystals show a >97.5% chemical purity. The enantiomeric excess is >97% ee by chiral HPLC. As SOL11476 has the same retention time as the (+)-enantiomer of (1S,5R)-(−)-1-(3,4-dichlorophenyl)-3-azabicyclo[3.1.0]hexane the exact value cannot be attributed. The material from this batch has −54.7° (c=2, MeOH) optical rotation.

Analysis:

### (1S,5R)-(−)-1-(3,4-DICHLOROPHENYL)-3-AZABICYCLO[3.1.0]HEXANE (free base)

¹H NMR (CDCl₃, 300 MHz) δ 7.34 (d, J=5.7 Hz, 1H), 7.27 (d, J=2.1 Hz, 1H), 7.03 (dd, J=5.7, 2.1 Hz, 1H), 3.25-3.00 (m, 4H, CH2NCH2), 1.74 (bs, 1H, NH), 1.74-1.65 (m, 1H. ArCCH2CH), 0.98-0.87 (m, 2H, ArCCH2CH).

### (1S,5R)-(−)-1-(3,4-DICHLOROPHENYL)-3-AZABICYCLO[3.1.0]HEXANE (HCl salt)

¹H NMR (CDCl₃, 300 MHz) δ 10.34 (bs, 1H, NH2Cl), 9.87 (bs, 1H, NH2Cl), 7.40 (d, J=8.1 Hz, 1H), 7.28 (d, J=2.1 Hz, 1H), 7.04 (dd, J=8.1, 2.1 Hz, 1H), 3.80-3.47 (m, 4H, CH2NCH2), 2.12-1.97 (m, 1H, ArCCH2CH), 1.66-1.62 (m, 1H, ArCCH2CH), 1.26-1.19 (m, 1H, ArCCH2CH).

¹³C-NMR (HCl salt) 138.62; 133.42; 132.08; 131.22; 1129.64; 126.94; 50.78; 47.83; 31.02; 23.90; 16.05.

HPLC: Rt=6.31 min

MS: m/z=229 [M+H]+

Optical Rotation:

54.7° (c=2 mg/ml MeOH)

### EXAMPLE III

### Preparation of 1-(3,4-dichlorophenyl)-3-azabicyclo[3.1.0]hexanes using Reaction Scheme 5

### A. Synthesis of 1-(3,4-dichlorophenyl)-2-hydroxymethyl-cyclopropanecarbonitrile

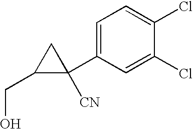

A 1000 mL triple neck flask was charged with dry THF (600 mL) and (3,4-dichlorophenyl)-acetonitrile (50 g, 271.7 mmol). The reaction was cooled to −25° C. and sodium amide (10.6 g, 271 mmol, 1 eq) was added in one portion. The reaction was allowed to warm to 20° C. over two hours. The reaction was then cooled again to −25° C. and epichlorohydrin (21 mL, 271 mmol) was added followed by sodium amide (10.5 g, 271 mmol, 1 eq). The reaction was slowly allowed to warm to 15° C. over 8 hrs. The reaction mixture was slowly added to saturated ammonia chloride (1000 mL) with stirring. The organic layer was separated, dried with magnesium sulfate and reduced to a dark yellow oil. Purification via biotage chromatography eluting with 1-5% Methanol in dichloromethane afforded 49.0 g, 75% yield (˜4-l_EE/ZZ) of a yellow oil. ¹H NMR (400 MHz, CHLOROFORM-d) d ppm 1.55-1.67 (m, 2H) 1.85-1.97 (m, 1H) 3.77 (dd, 1H) 4.09 (dd, 1H) 7.15 (dd, 1H) 7.31-7.47 (m, 2H).

### B. Synthesis of 1-(3,4-dichlorophenyl)-2-hydroxymethyl-cyclopropanecarbonitrile

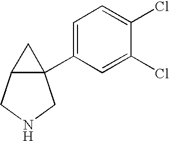

To a solution of 2-cyano-2-(3,4-dichlorophenyl)cyclopropanecarboxylic acid methyl ester (500 mg, 1.85 mmol) in dry tetrahydrofuran (50 mL) at 0° C. was added borane dimethylsulfide (3 mL; 2 M solution in THF). The reaction was heated to reflux for 3.5 hours. The reaction mixture was slowly cooled to 20-25° C. and slowly quenched into a 2N HCl solution (20 mL). The mixture was stirred at room temperature for 0.5 hours. The THF was removed by concentrating the mixture under vacuum using a rotary evaporator with a bath temperature of 35° C. Concentrated ammonium hydroxide (30 mL) and dichloromethane (100 mL) was then added, and the phases were stirred for 15 minutes before allowing them to separate. The aqueous phase was re-extracted with dichloromethane (1×40 mL). The combined organic layers were washed with brine (40 mL). The organic layer was dried over sodium sulfate, filtered and concentrated to an oil. The residue was taken up in dichloromethane (20 mL) and trifluoroacetic acid (1.5 mL) was added dropwise over a period of 15 minutes. The mixture was then concentrated to a residue using a rotary evaporator with a bath temperature of 30° C. The residue was slurried in diethyl ether (10 mL) for 30 minutes at room temperature, and the white solid product was filtered, rinsed with diethyl ether (5 mL), and dried under a nitrogen stream in vacuo to afford the desired product. (506 mg, 80% yield). ¹H NMR (400 MHz, DMSO-d₆) d ppm 1.08-1.19 (m, 1H) 1.38-1.50 (m, 2H) 2.12-2.18 (m, 1H) 3.32-3.37 (d, 1H) 3.42-3.54 (m, 2H) 3.58-3.71 (d, 1H) 7.07-7.27 (m, 1H) 7.40-7.60 (m, 2H).

### EXAMPLE IV

### Preparation of (+)-(1R,5S)-1-(3,4-dichlorophenyl)-3-ethyl-3-azabicyclo[3.1.0]hexane using Reaction Scheme 11

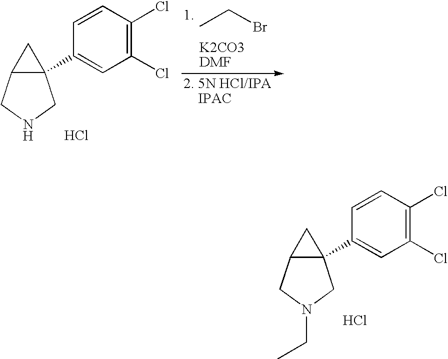

A 22 L four-necked round-bottomed-flask was equipped with a mechanical stirrer, bearing, stir shaft, paddle, thermowell, chart recorder, nitrogen inlet adapter and powder funnel. The flask was charged with (+)-1-(3,4-dichlorophenyl)-3-azabicyclo[3.1.0]hexane (1596.9 g, 5.8368 mol) and dimethylformamide (3.19 L). The mixture was agitated at ambient temperature for 10 minutes. Bromoethane (675 mL, 9.047 mol) and potassium carbonate (1250.4 g, 9.047 mol) were then added to the solution. The reaction was stirred at ambient temperature for 28 hours until 4.5% AUC of the starting material was present. The reaction was filtered through a sintered glass funnel and washed with heptane (6.38 L). The filtrate was transferred to the 22 L round bottom flask and the layers were stirred for a minimum of 5 minutes. The layers were allowed to partition for a minimum of 2 minutes, and the heptane layer containing the desired product was transferred to a carboy. The DMF layer was then extracted again with heptane (6.4 L). The combined heptane layers were washed with water (6.38 L). The organic layer was then dried over sodium sulfate (860.8 g) for 30 minutes, filtered, and the cake was rinsed with heptane (4 L). The filtrate was concentrated under vacuum using a rotary evaporator and a bath temperature of 40° C. to provide the desired free base as a yellow oil (1319.7 g).

The oil was transferred with the aide of isopropyl acetate (IPAC, 13.3 L) to a 22 L four-necked round-bottomed flask equipped with a mechanical stirrer, bearing, stir shaft, paddle, thermowell, chart recorder and nitrogen inlet adapter. To the stirred clear solution was added 5 to 6 N HCl in 2-propanol (1.236 L) at a rate to maintain the internal temperature below 30° C. to provide a white slurry. The slurry was cooled with stirring to 20° C. and stirred for 1 hour. The solids were collected on a tabletop funnel and rinsed with IPAC (2.65 L). The solids were then dried under vacuum at 50° C. to provide 1186.7 g (69.5%) of (+)-(1R)-1-(3,4-dichlorophenyl)-3-ethyl-3-azabicyclo[3.1.0]hexane as a white solid.

### EXAMPLE V

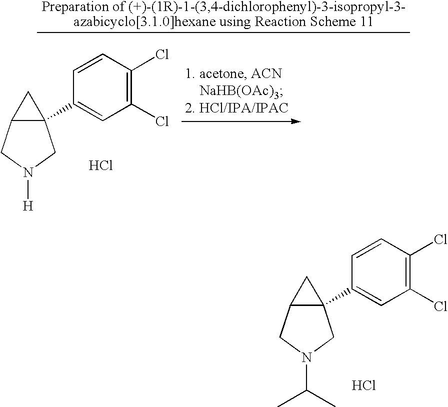

A 22 L four-necked round-bottomed-flask was equipped with a mechanical stirrer, bearing, stir shaft, paddle, thermowell, condenser, chart recorder, nitrogen inlet adapter and powder funnel. The flask was charged with (+)-1-(3,4-dichlorophenyl)-3-azabicyclo[3.1.0]hexane (1347.5 g, 4.925 mol), acetonitrile (3.8 L), and acetone (3.8 L). The resulting slurry was cooled to 0° C. and sodium triacetoxyborohydride (2706.7 g, 12.77 mol) was added in approximately 100 g portions over thirty-six minutes. The slurry warmed to 26° C. during the addition. The cold bath was removed once the batch cooled to 20° C. and the slurry was stirred at ambient temperature for 2 h until not more than 0.1% AUC of the starting material was present. A 50 L round bottom flask was charged with 5N NaOH (7.8 L) and isopropyl acetate {IPAC} (6.45 L). The reaction mixture was transferred to the stirring quench mixture in the 50 L flask using a transfer pump at a rate to maintain an internal temperature below 45° C. The mixture was then agitated for 10 minutes before the layers were allowed to separate. The lower aqueous phase was discarded. The organic phase was then washed with 5N NaOH (5.2 L), water for injection (2×6.45 L) and then half brine (6.1 L). With each extraction, the mixture was allowed to stir for a minimum of 5 minutes and a maximum of 15 minutes. The layers were allowed to separate for a minimum of 2 minutes. The organic phase was dried over magnesium sulfate (MgSO₄, 788.9 g) and filtered through a pad of Celite (4 cm). The Celite pad was then rinsed with IPAC (5.40 L) and the filtrates were transferred into a 22 L round bottom flask. To the stirred clear colorless solution was added 5 to 6 N HCl in 2-propanol (1.22 L) at a rate to maintain the internal temperature below 34° C. to provide a white slurry. The slurry was cooled with stirring to 20° C. over 0.5 h. The solids were collected on a tabletop funnel and rinsed with IPAC (5.37 L). The solids were then dried under vacuum at 50° C. for 22.5 h to provide 1367.8 g (90.6%) of (+)-(1R,5S)-1-(3,4-dichlorophenyl)-3-isopropyl-3-azabicyclo[3.1.0]hexane as a white solid.

### EXAMPLE VI

### Preparation of (1R,5S)-1-naphthalen-2-yl-3-azabicyclo[3.1.0]hexane hydrochloride using Reaction Schemes 1 & 12

### A. Synthesis of (1R,2S)-2-Hydroxymethyl-2-naphthyl-cyclopropancarbonitrile

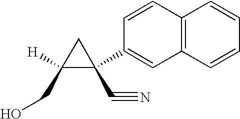

To a stirring solution of 2-naphthylacetonitrile (50.0 g, 0.299 moles) and (S)-(+)-epichlorohydrin (36.0 g, 0389 moles) in anhydrous THF (300 mL) at −15 to −20° C. under nitrogen, was added sodium his (trimethylsilyl)amide (2M in THF, 300 mL, 0.600 moles) slowly via addition funnel while keeping the temperature between −15° C. and −20° C. After completion of the addition, the mixture was stirred for 3 hours at −15° C. to −20° C. The reaction mixture was quenched by slow addition of 2M HCl (520 mL) allowing the temperature to rise to 15° C. as the neutralization proceeded. The layers were allowed to settle and the layers were separated. The aqueous layer was extracted once with ethyl acetate (300 mL). The organic portion was washed with brine (4000 mL) dried over sodium sulfate, filtered and concentrated under reduced pressure to provide an orange oil which was used without further purification.

### B. Synthesis of ((1S,2R)-2-Aminomethyl-2-naphthylen-2-yl cyclopropyl)-methanol

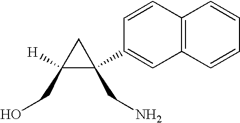

To a solution of nitrile in THF (300 mL) was slowly added borane dimethylsulfide (10 M, 60 mL, 0.60 moles) via addition funnel. The reaction temperature was maintained below 60° C. during the addition. After completion of the addition, the reaction was held at 60° C. until the starting nitrile was completely consumed (approximately 2.5 hours). The mixture was cooled below 15° C. and 2M HCl (200 mL) was slowly added maintaining a temperature below 20° C. The reaction mixture was then heated to 50° C. for one hour. After the heating period, the reaction was cooled below 30° C. and isopropyl acetate (200 mL) and water (250 mL) were added. The phases were separated and the organic phase was discarded. Ammonium hydroxide (75 mL) was added and the mixture cooled to 25° C. with stirring. The aqueous phase was extracted with isopropyl acetate (2×250 mL). The combined organic phases were washed with 5% dibasic sodium phosphate (200 mL) and saturated NaCl (200 mL), dried over sodium sulfate and concentrated. The viscous yellow oil was dissolved in isopropyl acetate (500 mL) and heated to 55° C. with stirring. p-Toluene sulfonic acid monohydrate (54.25 g, 0.285 mole) was added over 5 minutes. A white solid formed as the acid was added. The reaction mixture was slowly cooled to room temperature, filtered and washed with isopropyl acetate. Yielded—53.7 g white solid 45% (tosylate salt)

### C. Synthesis of (1R,5S)-1-naphthalen-2-yl-3-azabicyclo[3.1.0]hexane hydrochloride

To a stirring slurry of ((1S,2R)-2-aminomethyl-2-naphthylen-2-yl cyclopropyl)-methanol tosylate (53.7 g, 0.134 mole) in isopropyl acetate (350 mL), at room temperature under nitrogen, was added thionyl chloride (11.8 mL, 0.161 moles) slowly via addition funnel while keeping the temperature below 35° C. The resulting mixture was stirred for 1 hour, after which time, no starting material remained. The mixture was neutralized with the slow addition of 5 N NaOH (160 mL) keeping the temperature below 30° C. The phases were separated and the aqueous phase was extracted with isopropyl acetate (200 mL). The combined organic extracts were washed with saturated sodium chloride (150 mL), dried over sodium sulfate, filtered and concentrated to 300 mL. The hydrochloride was made directly from this solution by slowly adding HCl in 2-propanol (5-6N, 26 mL). The mixture was stirred for 15 minutes and filtered and washed with isopropyl acetate. The wet cake was slurried in 2-propanol (400 mL) and heated to reflux with stirring under nitrogen for 2 hours. The resulting slurry was allowed to cool and stir at room temperature overnight. The resulting slurry was filtered and washed with 2-propanol. The solid was dried in a vacuum oven at 40° C. Yield—21.1 g, 64.2% ¹H NMR (400 MHz, DMSO-d₆) d ppm 1.23 (t) 1.40 (t) 2.21-2.28 (m) 3.40-3.47 (m) 3.50-3.66 (m) 3.74-3.82 (m) 7.39 (dd) 7.44-7.55 (m) 7.82 (s) 7.84-7.92 (m) 9.33 (br. s.) 9.69 (br. s.). LC/MS (m/z M⁺¹ 210)

It will be understood that the instant invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

Armarego, W. L. F. et. al., J. Chem. Soc. [Section C: Organic] 19: 3222-3229 (1971)
Szalecki et al., patent publication PL 120095 B2, CAN 99:158251
Cabadio, S. et al., Fr. Bollettino Chimico Farmaceutico 117: 331-42 (1978)
Mouzin, G. et al., Synthesis 4: 304-305 (1978)
“Nitrogen Protecting Groups in Organic Synthesis”, John Wiley and Sons, New York, N.Y., 1981, Chapter 7
“Nitrogen Protecting Groups in Organic Chemistry”, Plenum Press, New York, N.Y., 1973, Chapter 2
Green, T. W. and Wuts, P. G. M. in “Protective Groups in Organic Chemistry”, 3rd edition, John Wiley & Sons, New York, N.Y., 1999
U.S. Pat. No. 6,569,887; May 27, 2003; Lippa and Epstein
U.S. Pat. No. 6,716,868; Apr. 6, 2004; Lippa and Epstein
U.S. Pat. No. 4,231,935; Nov. 4, 1980; Fanshawe et al.

What is claimed is:

1. A method for making (1S,5R)-(−)-1-(3,4-dichlorophenyl)-3-azabicyclo[3.1.0]hexane comprising:
(a) reacting a compound of the following formula (i), 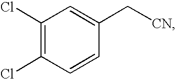
with (R)-(−)-epichlorohydrin to produce a compound of the following formula (ii), 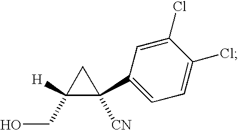
(b) reducing the compound of the formula (ii) to produce a compound of the following formula (iii), 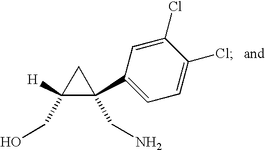
(c) causing cyclization of the compound of formula (iii) to produce (1S,5R)-(−)-1-(3,4-dichlorophenyl)-3-azabicyclo[3.1.0]hexane.

2. The method according to claim 1 further comprising:
(d) converting the (1S,5R)-(−)-1-(3,4-dichlorophenyl)-3-azabicyclo[3.1.0]hexane to a pharmaceutically acceptable salt.

3. The method according to claim 1 wherein in step (b) the compound of formula (ii) is reduced using lithium aluminum hydride, sodium aluminum hydride, NaBH₄ with ZnCl₂ or catalytic hydrogenation.

4. The method according to claim 1 wherein in step (b) the compound of formula (ii) is reduced using lithium aluminum hydride.

5. The method according to claim 1 wherein in step (c) the cyclization of the compound of formula (iii) is performed using SOCl₂ or POCl₃.

6. The method according to claim 1 wherein in step (c) the cyclization of the compound of formula (iii) is performed using SOCl₂.

7. The method according to claim 2 wherein the pharmaceutically acceptable salt is the hydrochloride salt.

8. A compound selected from the group consisting of compounds having the following formulas:

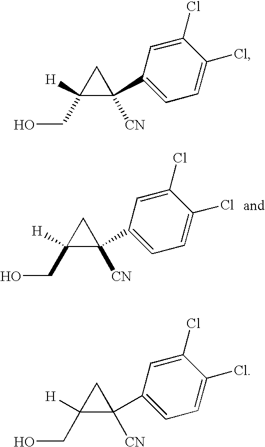

9. A compound selected from the group consisting of compounds having the following formulas and pharmaceutically acceptable salts thereof:

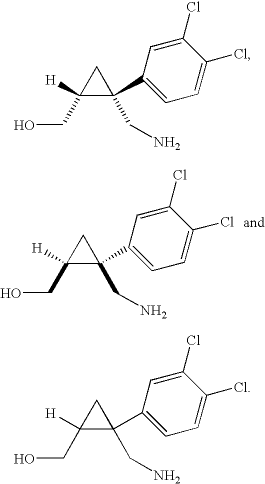

10. A method for making (1S,5R)-(−)-1-(3,4-dichlorophenyl)-3-azabicyclo[3.1.0]hexane comprising:
(a) reacting a compound of the following formula (i), 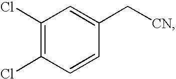
with
(R)-(−)-epichlorohydrin to produce a compound of the following formula (ii), 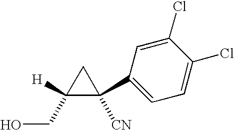
(b) reducing the compound of the formula (ii) to produce a compound of the following formula (iii) 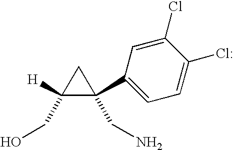
(c) reacting the compound of the formula (iii) with (Boc)₂O to produce a compound of the following formula (iv), 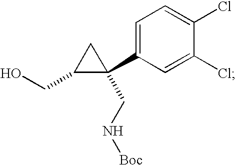
(d) deprotecting and causing cyclization of the compound of the formula (iv) to produce a compound of the following formula (v) 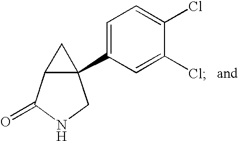
(e) reducing the compound of the formula (v) to produce (1S,5R)-(−)-1-(3,4-dichlorophenyl)-3-azabicyclo[3.1.0]hexane.

11. The method according to claim 10 further comprising:
(f) converting the (1S,5R)-(−)-1-(3,4-dichlorophenyl)-3-azabicyclo[3.1.0]hexane to a pharmaceutically acceptable salt.

12. The method according to claim 10 wherein in step (b) the compound of formula (ii) is reduced using lithium aluminum hydride, sodium aluminum hydride, NaBH₄ with ZnCl₂ or catalytic hydrogenation.

13. The method according to claim 10 wherein in step (b) the compound of formula (ii) is reduced using lithium aluminum hydride.

14. The method according to claim 10 wherein in step (d) the cyclization is performed using TFA.

15. The method according to claim 10 wherein in step (e) the compound of formula (v) is reduced using lithium aluminum hydride, sodium aluminum hydride, NaBH₄ with ZnCl₂ or catalytic hydrogenation.

16. The method according to claim 11 wherein the pharmaceutically acceptable salt is the hydrochloride salt.

17. A compound having the formula:

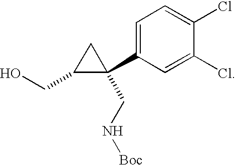

18. A compound having the formula:

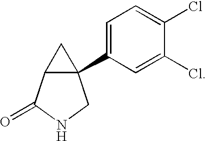

19. A method of making a 1-aryl-3-azabicyclo[3.1.0]hexane of the following formula I, 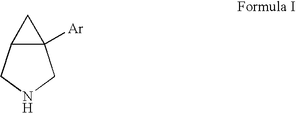
wherein Ar is a phenyl or napthyl group which is either unsubstituted or substituted with one or more substituents independently selected from halogen, C₁₋₃ alkyl, C₂₋₄ alkenyl, C₂₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino, comprising the steps of:
(a) reacting a compound of the following formula v(i), 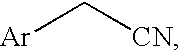
wherein Ar is defined as above, with epichlohydrin or an enantiomer thereof, to produce a compound of the following formula (vii), 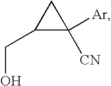
or an enantiomer or diastereomer thereof;
(b) oxidizing the compound of formula (vii) to produce a compound of the following formula (viii), 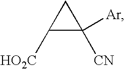
or an enantiomer or diastereomer thereof;
(c) causing esteration of the compound of formula (viii) to produce a compound of the following formula (ix), 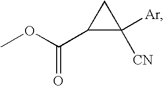
or an enantiomer or diastereomer thereof; and
(d) reducing the compound of formula (ix) to produce the 1-aryl-3-azabicyclo[3.1.0]hexane, or an enantiomer or diastereomer thereof.

20. The method according to claim 19 further comprising:
(e) converting the 1-aryl-3-azabicyclo[3.1.0]hexane, or an enantiomer or diastereomer thereof, to a pharmaceutically acceptable salt.

21. The method according to claim 19 wherein the epichlorohydrin or enantiomer thereof is S-(+)-epichlorohydrin and the 1-aryl-3-azabicyclo[3.1.0]hexane, or the enantiomer or diastereomer thereof, is (1R,5S)-(+)-1-aryl-3-azabicyclo[3.1.0]hexane.

22. The method according to claim 19 wherein the epichlorohydrin or enantiomer thereof is R-(−)-epichlorohydrin and the 1-aryl-3-azabicyclo[3.1.0]hexane, or the enantiomer or diastereomer thereof, is (1S,5R)-(−)-1-aryl-3-azabicyclo[3.1.0]hexane.

23. The method according to claim 19 wherein Ar is 3,4-dichlorophenyl and the 1-aryl-3-azabicyclo[3.1.0]hexane, or the enantiomer or diastereomer thereof, is 1-(3,4-dichlorophenyl)-3-azabicyclo[3.1.0]hexane, or an enantiomer or diastereomer thereof.

24. The method according to claim 23 wherein the epichlorohydrin or enantiomer thereof is R-(−)-epichlorohydrin and the 1-(3,4-dichlorophenyl)-3-azabicyclo[3.1.0]hexane, or the enantiomer or diastereomer thereof, is (1S,5R)-(−)-1-(3,4-dichlorophenyl)-3-azabicyclo[3.1.0]hexane.

25. The method according to claim 19 wherein the epichlorohydrin or enantiomer thereof is S-(+)-epichlorohydrin and the 1-(3,4-dichlorophenyl)-3-azabicyclo[3.1.0]hexane, or the enantiomer or diastereomer thereof, is (1R,5S)-(−)-1-(3,4-dichlorophenyl)-3-azabicyclo[3.1.0]hexane.

26. A method of making 1-(3,4-dichlorophenyl)-3-azabicyclo[3.1.0]hexane comprising:
(a) reacting a compound of the following formula (i), 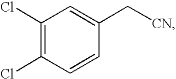
with epichlohydrin or an enantiomer thereof, to produce a compound of the following formula (x), 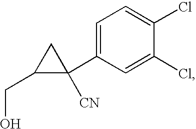
or an enantiomer or diastereomer thereof;
(b) oxidizing the compound of formula (x) to produce a compound of the following formula (xi), 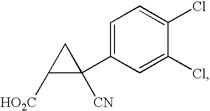
or an enantiomer or diastereomer thereof;
(c) causing esteration of the compound of formula (xi) to produce a compound of the following formula (xii), 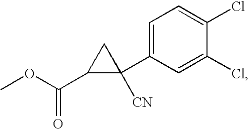
or an enantiomer or diastereomer thereof;
(d) reducing the compound of formula (xii) in the presence of a Raney nickel catalyst to produce a compound of the following formula (xiii), 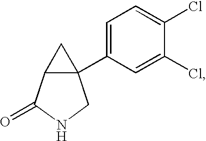
or an enantiomer or diastereomer thereof; and
(e) reducing the compound of formula (xiii) to produce the 1-(3,4-dichlorophenyl)-3-azabicyclo[3.1.0]hexane, or an enantiomer or diastereomer thereof.

27. The method according to claim 26 further comprising:
(e) converting the 1-aryl-3-azabicyclo[3.1.0]hexane, or an enantiomer or diastereomer thereof, to a pharmaceutically acceptable salt.

28. The method according to claim 26 wherein the epichlorohydrin or enantiomer thereof is R-(−)-epichlorohydrin and the 1-(3,4-dichlorophenyl)-3-azabicyclo[3.1.0]hexane, or the enantiomer or diastereomer thereof, is (1S,5R)-(−)-1-(3,4-dichlorophenyl)-3-azabicyclo[3.1.0]hexane.

29. The method according to claim 26 wherein the epichlorohydiin or enantiomer thereof is S-(+)-epichlorohydrin and the 1-(3,4-dichlorophenyl)-3-azabicyclo[3.1.0]hexane, or the enantiomer or diastereomer thereof, is (1R,5S)-(−)-1-(3,4-dichlorophenyl)-3-azabicyclo[3.1.0]hexane.

30. A compound selected from the group consisting of compounds having the following formulas and pharmaceutically acceptable salts thereof: 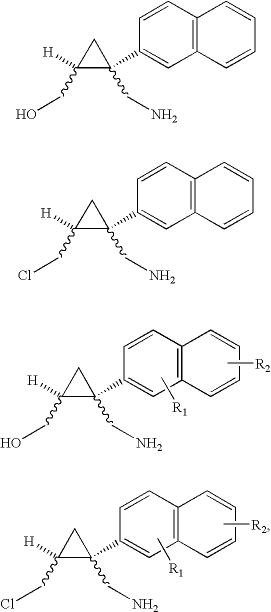
wherein R₁ and R₂ are independently selected from hydrogen, halogen, C₁₋₃ alkyl, C₂₋₄ alkenyl, C₁₋₄ alkynyl, halo(C₁₋₃)alkyl, cyano, hydroxy, C₃₋₅ cycloalkyl, C₁₋₃ alkoxy, C₁₋₃ alkoxy(C₁₋₃)alkyl, carboxy(C₁₋₃)alkyl, C₁₋₃ alkanoyl, halo(C₁₋₃)alkoxy, nitro, amino, C₁₋₃ alkylamino, and di(C₁₋₃)alkylamino.
